(19) United States
(12) Patent Application Publication
(10) Pub. No.: US 2011/0041846 A1
(43) Pub. Date: Feb. 24, 2011

(54) LIQUID HOLDER, AND INHALATION APPARATUS EMPLOYING THE SAME

(75) Inventors: Kazuo Kusakabe, Tokyo (JP); Masaru Sugita, Tokyo (JP); Masaya Kobayashi, Yokohama-shi (JP)

Correspondence Address: FITZPATRICK CELLA HARPER & SCINTO, 1290 Avenue of the Americas, NEW YORK, NY, 10104-3800, US

(73) Assignee: CANON KABUSHIKI KAISHA, Tokyo (JP)

(21) Appl. No.: 12/990,518

(22) PCT Filed: Jul. 17, 2009
(86) PCT No.: PCT/JP2009/063301

§ 371 Date: Nov. 1, 2010

(30) Foreign Application Priority Data
Jul. 18, 2008 (JP) 2008-186810

(51) Int. Cl. A61M 15/00; B65D 90/32

(52) U.S. Cl. 128/203.12; 220/89.1

(57) ABSTRACT

A liquid holder for holding a liquid comprises an outlet formation part for formation of outlet port for discharging the liquid held in the liquid holder, and a pressure differential-reducing member for reducing a predetermined pressure differential between the inside and outside of the liquid holder; the pressure differential-reducing member including a first member which moves for reducing the first predetermined pressure differential and a second member which reduces a second pressure differential less than the first predetermined pressure differential.

### TECHNICAL FIELD

The present invention relates to a liquid holder for holding a liquid such as a medical solution, and an inhalation apparatus employing the liquid holder.

### BACKGROUND ART

Inhalation apparatuses have been developed for inhalation of fine droplets of a medical solution, through a mouthpiece, based on the principle of an inkjet system (Japanese Patent Application Laid-Open Nos. 2004-290593 and 2004-283245). Such an inhalation apparatus is capable of spraying precisely a prescribed amount of a medical solution in a uniform particle size.

Such a medical solution-ejecting apparatus (liquid droplet ejecting apparatus) comprises, as basic constitution elements, an ejection head having an ejection energy-generating element like a heater element, and a medical solution tank for holding the medical solution. With a medical solution tank of a simple closed structure, with decrease of the amount of the medical solution in the tank by ejection of the liquid, the pressure in the tank becomes negative, resulting in lower ejection performance. To prevent the lowering of the ejection performance, countermeasures are taken as mentioned below.

As one countermeasure, the medical solution tank is allowed to communicate with the outside air immediately before the start of the liquid ejection. This air communication is employed in conventional inkjet printers. However, with an inhalation apparatus in which the medical solution is stored in an amount for plural times of inhalation, the tank should be completely air-tight, so that the communication to the outside air is not employed in view of prevention of concentration change or denaturation of the medical solution. This is true in the case of a medical solution which is sensible to the air.

To prevent the contact of the medical solution with the air, for example, the main body of the tank is made from a glass, and the one open end thereof is closed by a plug (e.g., a rubber plug) which is slidable freely in correspondence with consumption of the liquid by ejection to decrease the volume of the tank. Specifically, as illustrated in FIG. 21, glass-made liquid-holder 201 containing liquid 205 is closed at one end by stopper 202, and the other open end is closed by a movable plug 209 made of rubber to seal the liquid 205. With this liquid holder 201, movable plug 209 moves into liquid holder 201 with ejection of liquid 205 to reduce the negative pressure when the negative pressure in liquid holder 201 exceeds the prescribed level. In FIG. 21, the numeral 203 denotes the main body of the reservoir of the holder (e.g., made of glass). Ejection head 206 having communication needle 208 is placed in opposition to stopper 202. Ejection head 206 has ejection outlet 207 for ejecting liquid 205.

With the highly air-tight liquid holder like that mentioned above, with progress of ejection of the medical solution, the pressure differential (atmospheric pressure differential) between the inside and outside of the liquid tank increases. For movement of the rubber plug (movable plug) to reduce the negative pressure, a considerable pressure difference is necessary. The movable plug starts to move when the force applied to the movable plug by the negative pressure in the liquid tank exceeds the maximum frictional force between the glass-made holder and the movable plug. Thus, when the movable plug is fit to press hard the glass-made holder wall to keep sufficiently the air-tightness, the force corresponding thereto is required for the movement of the movable plug.

On the other hand, increase of the negative pressure in the medical solution tank will lower the performance of ejection from the ejection head. For example, in ejection through a nozzle of 3 μm diameter, the rate of the ejection can be kept unchanged before the internal pressure comes to be −5 kPa, but decreases gradually at the higher negative pressure, the ejection being interrupted at an internal pressure of −20 kPa by sucking the outside air though the ejection head reversely. Therefore, for stable ejection of the medical solution, the negative pressure in the liquid tank is kept preferably less than the prescribed level (−5 kPa in the above example).

However, a usual highly air-tight liquid holder like that mentioned above can not easily keep the negative pressure in the liquid holder to be less than the prescribed level, causing drop of the ejection performance, or failure of the ejection.

### DISCLOSURE OF THE INVENTION

To overcome the above disadvantages, the present invention intends to provide a liquid holder which is capable of decreasing the negative pressure caused during ejection of a liquid enclosed in a liquid holder not to affect adversely the ejection performance, and intends also to provide an inhalation apparatus equipped with the liquid holder.

The present invention is directed to a liquid holder for holding a liquid comprising:

an outlet formation part for formation of outlet port for discharging the liquid held in the liquid holder, and a pressure differential-reducing member for reducing a first predetermined pressure differential between the inside and outside of the liquid holder;

the pressure differential-reducing member including a first member which moves for reducing the first predetermined pressure differential and a second member which reduces a second pressure differential less than the first predetermined pressure differential.

The first member and the second member can be formed in one body, and move together when reducing the first predetermined pressure differential, the second member deforms to reduce the second pressure differential less than the first predetermined pressure differential.

The first member and the second member can be connected by an expandable connector and move together when reducing the first predetermined pressure differential, and

the second member reduces the second pressure differential less than the first predetermined pressure differential by changing the distance from the first member.

The first member can have an air hole for communicating a gap between the first member and the second member with the outside of the liquid holder.

The pressure differential-reducing member can have a recovery means for bringing the second member to be ready for reducing the second pressure differential less than the first predetermined pressure differential at the time when the first predetermined pressure differential has been reduced by movement of the first member.

The pressure differential-reducing member can have a position-limiter for limiting the range of displacement of the first member or the second member.

The present invention is directed to an inhalation apparatus, comprising

a liquid holder set forth in any of claims 1 to 6, an ejection head for ejecting a liquid held in the liquid holder, and

a suction port for inhalation of the liquid ejected from the ejection head by a user.

According to the present invention, the liquid holder has a second member for reducing the second pressure differential of less than a prescribed first pressure differential between the inside and outside of the liquid holder, which enable control of the increase of the negative pressure in the process of ejection of the liquid in a tightly closed state not to adversely affect the ejection performance.

Other features and advantages of the present invention will be apparent from the following description taken in conjunction with the accompanying drawings, in which like reference characters designate the same or similar parts throughout the figures thereof.

### BRIEF DESCRIPTION OF THE DRAWINGS

FIGS. 1A, 1B and 1C illustrate schematically a constitution of the liquid holder of First Embodiment. FIG. 1A is a schematic drawing of the constitution before connection of an ejection head. FIG. 1B illustrates schematically the constitution at a first state with the ejection head attached. FIG. 1C illustrates schematically the constitution at a second state with the ejection head attached.

FIGS. 2A and 2B are graphs showing change with time of the pressure in the liquid holder in the course of ejection of the liquid. FIG. 2A is a graph of the pressure change during the ejection with a liquid holder of the present invention. FIG. 2B is a graph of the pressure change in the course of the ejection with a conventional liquid holder.

FIGS. 3A, 3B and 3C illustrate a movable plug of Modification Example 1. FIG. 3A is a sectional view of the movable plug in a normal state. FIG. 3B is a sectional view of the movable plug in a first deformation state. FIG. 3C is a sectional view of the movable plug in a second deformation state.

FIGS. 4A, 4B, 4C and 4D illustrate a movable plug of Modification Example 2. FIG. 4A is a sectional view of the movable plug in a normal state. FIG. 4B is a sectional view of the movable plug in a first deformation state. FIG. 4C is a sectional view of the movable plug in a second deformation state. FIG. 4D is a side view of the movable plug taken from the right side in FIG. 4A.

FIGS. 5A, 5B and 5C illustrate a movable plug of Modification Example 3. FIG. 5A is a sectional view of the movable plug having a spacer inserted into the hollow of the main sliding portion. FIG. 5B is a side view of the movable plug taken from the right side in FIG. 5A. FIG. 5C is a sectional view of another movable plug.

FIGS. 6A and 6B are sectional view of another main sliding portion (of the movable plug).

FIGS. 7A, 7B, 7C and 7D illustrate a movable plug of Modification Example 4. FIG. 7A is a sectional view of the movable plug in a normal state. FIG. 7B is a sectional view of the movable plug in a first deformation state. FIG. 7C is a sectional view of the movable plug in a second deformation state. FIG. 7D is a side view of the movable plug taken from the right side in FIG. 7A.

FIGS. 8A, 8B and 8C illustrate a movable plug of Modification Example 5. FIG. 8A is a sectional view of the movable plug in a normal state. FIG. 8B is a sectional view of the movable plug in a first deformation state. FIG. 8C is a sectional view of the movable plug in a second deformation state.

FIGS. 9A, 9B, 9C and 9D illustrate a movable plug of Modification Example 6. FIG. 9A is a sectional view of the movable plug in a normal state. FIG. 9B is a sectional view of the movable plug in a first deformation state. FIG. 9C is a sectional view of the movable plug in a second deformation state. FIG. 9D is a side view of the movable plug taken from the right side in FIG. 9A.

FIGS. 10A, 10B, 10C and 10D illustrate a movable plug of Modification Example 7. FIG. 10A is a sectional view of the movable plug in a normal state. FIG. 10B is a sectional view of the movable plug in a first deformation state. FIG. 10C is a sectional view of the movable plug in a second deformation state. FIG. 10D is a side view of the movable plug taken from the right side in FIG. 10A.

FIG. 11 is a perspective view of a medical solution-inhalation apparatus employing a liquid holder of the present invention for inhalation of the medical solution by a user.

FIG. 12 is a perspective view of the inhalation apparatus of FIG. 11 with the access cover opened.

FIGS. 13A, 13B and 13C illustrate schematically the constitution of the liquid holder in Second Embodiment. FIG. 13A is a schematic drawing before connection of an ejection head. FIG. 13B illustrates schematically a first state after connection of the ejection head. FIG. 13C illustrates schematically a second state after connection of the ejection head.

FIG. 14 is a graph showing change with time of the pressure in the liquid holder in the course of ejection of the liquid.

FIGS. 15A, 15B and 15C illustrate schematically the constitution of the liquid holder in another Modification Example 1. FIG. 15A is a schematic sectional view of the liquid holder in a normal state. FIG. 15B is a sectional view of the second reservoir in a first state. FIG. 15C is a sectional view of the second reservoir in a second state.

FIG. 16 is a graph showing change with time of the pressure in the liquid holder in the course of ejection of the liquid.

FIGS. 17A, 17B and 17C illustrate schematically the constitution of the liquid holder in another Modification Example 2. FIG. 17A is a schematic sectional view of the liquid holder in a normal state. FIG. 17B is a sectional view of the second reservoir in a first state. FIG. 17C is a sectional view of the second reservoir in a second state.

FIGS. 18A, 18B and 18C illustrate schematically the constitution of the liquid holder in another Modification Example 3. FIG. 18A is a schematic sectional view of the liquid holder in a normal state. FIG. 18B is a sectional view of the second reservoir in a first state. FIG. 18C is a sectional view of the second reservoir in a second state.

FIG. 19 is a graph showing change with time of the pressure in the liquid holder in the course of ejection of the liquid.

FIG. 20 is a sectional view of a liquid holder of another Modification Example 4.

FIG. 21 is a sectional view of a conventional liquid holder to be compared with the one of the present invention.

### BEST MODE FOR CARRYING OUT THE INVENTION

Preferred embodiments of the present invention will now be described in detail in accordance with the accompanying drawings.

### First Embodiment

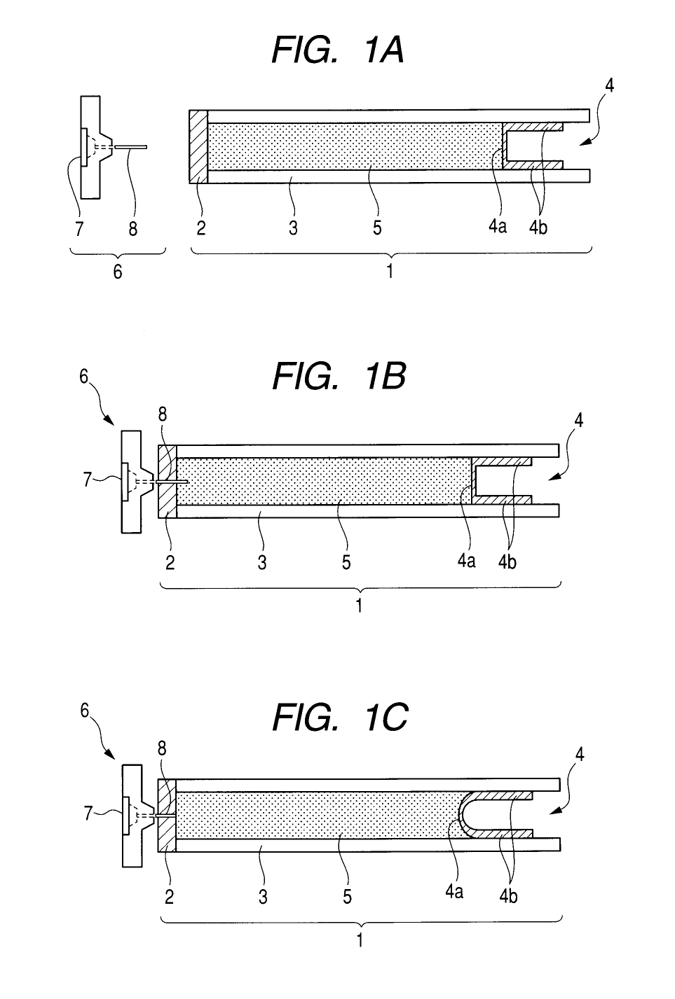

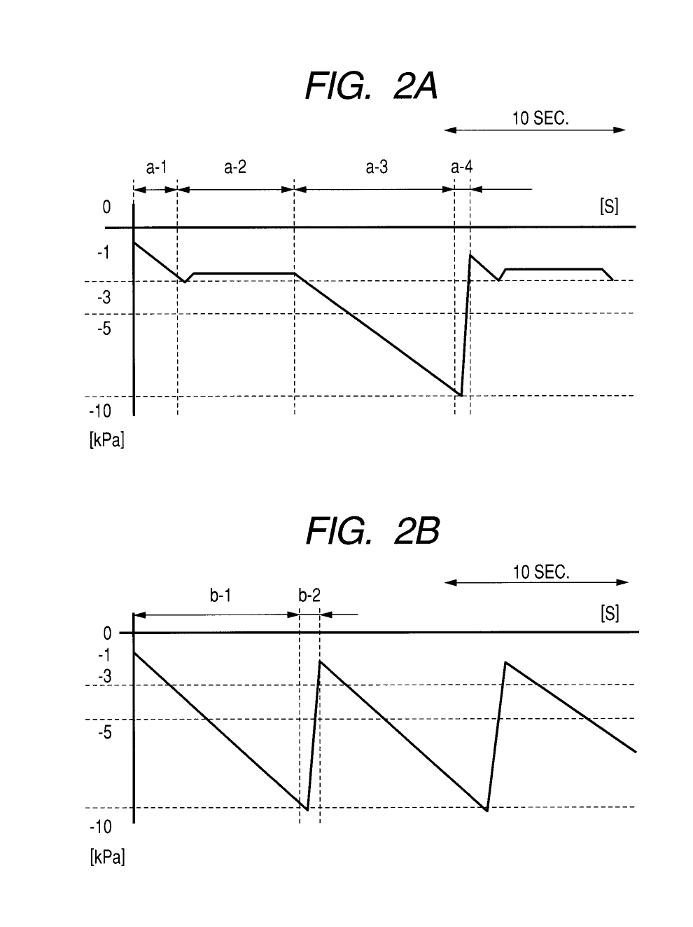

Liquid holder 1 of First Embodiment of the present invention is described with reference to FIGS. 1A to 1C and FIGS. 2A and 2B. FIGS. 1A to 1C illustrates schematically the constitution of the liquid holder of First Embodiment. FIG. 1A is a schematic drawing of the constitution before connection of ejection head 6. FIG. 1B illustrates schematically the constitution at a first state with ejection head 6 attached. FIG. 1C illustrates schematically a second state after connection of ejection head 6. FIGS. 2A and 2B are graphs showing change with time of the pressure in the liquid holder in the course of ejection of the liquid. FIG. 2A is a graph of the change in the course of the ejection with liquid holder 1 of the present invention. FIG. 2B is a graph of the pressure change in the course of the ejection with a conventional liquid holder.

Liquid holder 1 comprises reservoir 3 as the main body, plug (outlet-formation part) 2, and movable plug (pressure differential reducer) 4 as illustrated in FIGS. 1A to 1C. Reservoir 3 is made of a rigid material (e.g., glass) in a cylindrical shape. Stopper 2 closes the lengthwise end of cylindrical reservoir 3, and movable plug 4 closes the other lengthwise end to enclose liquid 5 in corporation with stopper 2. This movable plug 4 is constituted of membrane 4a (second member) characteristic of the present invention as illustrated in FIG. 1A and main sliding portion (first member) 4b for supporting the membrane 4a and formed in integration with membrane 4a. Liquid holder 1 encloses liquid 5 by utilizing reservoir 3, stopper 2, and movable plug 4.

Ejection head 6 having communication needle 8 is connectible to liquid holder 1 by inserting communication needle 8 at the position confronting the stopper 2 outside liquid holder 1. Ejection head 6 connected to liquid holder 1 can eject liquid 5 contained in liquid holder 1 through ejection outlet 7. Thus, ejection outlet 7 for ejecting liquid 5 is connected to stopper 2.

Ejection head 6 has an ejection energy-generating element (not shown in the drawing) near ejection outlet 7 for generating the energy for ejection of liquid 5. This energy-generating element gives ejection energy to the liquid delivered through communication needle 8 to eject the liquid from ejection outlet 7. The type of the ejection energy-generating element is not limited, and exemplified by electrothermal conversion elements for giving thermal energy to the liquid, and electromechanical conversion elements for giving mechanical energy to the liquid. Thus, the system of the liquid ejection includes a thermal jet system which ejects the liquid by giving thermal energy to the liquid by an electrothermal conversion element, and a piezo-jet system which ejects the liquid by utilizing pressure vibration by an electrothermal conversion element (e.g., piezo-element) for giving mechanical energy to the liquid. The liquid ejection system is selected to be suitable for the kind of the liquid to be ejected.

With the above thermal jet system, the liquid droplet size distribution can be narrowed by raising the precision and reproducibility of the ejection head, including the ejection outlet diameters, the heat quantity of the thermal pulses for the ejection, size of the micro-heater as the electrothermal conversion elements. The heads of thermal jet system is produced at a low cost and is suitable for a small-sized apparatus which requires frequent exchange of the head. Therefore, the thermal jet type system is particularly preferred for application of the liquid holder of the present invention to an inhalation apparatus for portability and convenience.

With ejection head 6 connected to liquid holder 1, liquid 5 is isolated from the outside air except at the ejection outlet 7. Therefore, the decrease of the amount of liquid 5 by ejection of the liquid from the ejection outlet 7 causes a pressure differential between the outside and inside of liquid holder 1. When the pressure differential has reached a prescribed level (described later with reference to FIGS. 2A and 2B) movable plug 4 moves into liquid holder 1 (leftward in FIGS. 1A to 1C to reduce the pressure differential to decrease the inside volume of liquid holder 1. The prescribed pressure differential mentioned above corresponds to the maximum frictional force at the site of contact of movable plug 4 with the inside wall of reservoir 3. Thus, when the pressure differential caused between the inside and outside of liquid holder 1 is less than the prescribed level for the movement of movable plug 4, membrane 4a is deformed to reduce the pressure differential.

The operation of ejection with liquid holder 1 mentioned above is described with reference to FIGS. 2A and 2B. In FIGS. 2A and 2B, the abscissa indicates the time, and the ordinate indicates the pressure differential between the inside and outside of the holder.

The operation of the ejection with liquid holder 1 can be considered for the time periods of (a-1), (a-2), (a-3), and (a-4). The ejection operation is described below for the time periods of (a-1) to (a-4).

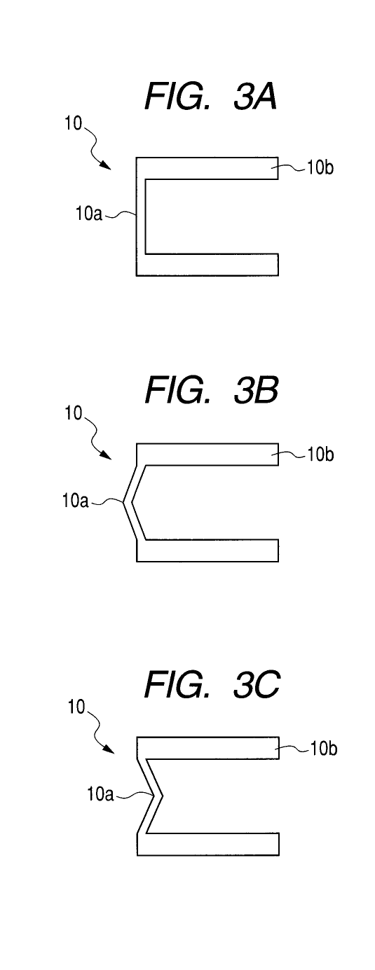

Before the ejection, the internal pressure in liquid holder 1 is preferably to be suitable for the ejection, ranging specifically from about −1 kPa to −3 kPa. If the internal pressure in liquid holder 1 becomes positive, the liquid tends to leak out from liquid holder 1, whereas if the internal pressure is excessively negative, the ejection of the liquid is abnormal. In this embodiment, the internal pressure in liquid holder 1 immediately before the ejection is selected to be at −1 kPa. With progress of the ejection of the liquid, the amount of the liquid in liquid holder 1 decreases to make the internal pressure more negative (period (a-1)). By use of a liquid holder in Modification Example 1 mentioned later with reference to FIGS. 3A to 3C, ejection of about 50 μL lowers the internal pressure to −3 kPa.

When the internal pressure in liquid holder 1 becomes lower than −3 kPa, membrane 4a begins to deform. Further ejection makes the membrane deform further, while the internal pressure in liquid holder 1 is kept at −3 kPa (period (a-2)). However, still further repetition of the ejection deforms membrane 4a for reducing the pressure differential to the deformation limit (boundary between period (a-2) and period (a-3)).

Beyond the deformation limit of the membrane 4a, the internal pressure in liquid holder 1 decreases at the same rate as in period (a-1) as shown in FIG. 2A (a-3). When the internal pressure in liquid holder 1 becomes lower than the threshold pressure (−10 kPa in this Example) for initiation of the movement of movable plug 4, movable plug 4 starts to move. This movement of movable plug reduces the pressure differential between the inside and outside of liquid holder 1 (period (a-4)). This movement of plug 4 stops when the force applied to the movable plug 4 by the negative pressure becomes smaller than the dynamic frictional force between liquid holder 1 and movable plug 4.

Next, the ejection operation of conventional liquid holder 201 (FIG. 21) is described for comparison with the present invention. The operation of the ejection with liquid holder 201 can be considered for the time periods of (b-1) and (b-2) as indicated at the upper portion of FIG. 2B.

Movable plug 209 of liquid holder 201 does not have a member like the membrane 4a which is characteristic of the present invention. Therefore, the pressure differential between the inside and outside of liquid holder 201 increases to the level (−10 kPa similarly as in FIG. 2A) for starting the movement of movable plug 209 at the rate indicated in the graph in period (b-1) as illustrated in FIG. 2A.

When the internal pressure in liquid holder 201 exceeds the pressure differential for starting the movement of movable plug 209, movable plug 209 starts the movement to reduce the pressure differential between the inside and outside of liquid holder 201 until movable plug 209 stops (period (b-2)). Movable plug 209 stops when the force applied to movable plug 209 by the negative pressure in liquid holder 201 becomes weaker than the dynamic frictional force between liquid holder 201 and movable plug 209.

The processes of FIG. 2A and FIG. 2B are compared below. In period (a-2), the internal pressure in liquid holder 1 is kept relatively low (about −3 kPa in this example). In contrast, in period (b-1) corresponding to period (a-2), the internal pressure in liquid holder 201 becomes gradually more negative from −3 kPa to the final level of −10 kPa. Thus, with liquid holder 1 of this embodiment, the internal pressure is kept during the time periods (a-1) and (a-2) not to lower the ejection performance. In other words, liquid holder 1 of this Embodiment can maintain the internal pressure at the level not to lower the ejection performance during the time periods (a-1) and (a-2), whereas with liquid holder 201, the internal pressure becomes lower to the level of lowering the ejection performance within the period (b-1).

Therefore, for example, for ejection of a medical solution for one administration by inhalation over a period (a-2), liquid holder 1 of this Embodiment is suitable which is capable of keeping the pressure differential at about −3 kPa during period (a-2). Before the next inhalation the starting internal pressure in liquid holder 1 can be set equal for every inhalation by recovering the initial state of movable plug 4. The recovery of the initial state herein signifies that the movable plug 4 is forcibly slided into liquid holder 1 (leftward in FIGS. 1A to 1C) to bring the internal pressure to the initial state of about −1 kPa and to cancel the deformation of membrane 4a.

In the above example, the first threshold level is set at −3 kPa for reducing the pressure differential by deformation (deflection) of membrane 4a, and the second threshold level is set at −10 kPa for reducing the pressure differential by movement of the entire movable plug 4. However, the threshold levels may be set at arbitrary levels without limitation. The first threshold level can be adjusted suitably by the thickness and material of membrane 4a, and the second threshold level can be adjusted by the size and material of movable plug 4.

The kind of liquid 5 is not limited specially. For use of liquid holder 1 of the present invention for an inhalation apparatus, liquid 5 may be a medical solution for medical treatment. The medical solution includes not only liquids of pharmaceutically active and physiologically active medical compounds but also liquids for charming tastes or charming perfumes, liquids of dyes, pigments and so forth. Further the medical solution may contain an additive.

The constitution material of reservoir 3 as the main body of liquid holder 1 includes, in addition to glass, resins such as polycarbonate resins, ABS resins, cycloolefin resins, and methacryl resins, and complex resins such as polyethylene/(ethylene-vinyl alcohol copolymer), and polypropylene/(ethylene-vinyl alcohol copolymer).

The material of movable plug 4 and membrane 4a includes butyl rubber, and isoprene rubber. The material is selected in consideration of the stability to liquid 5 and elution into the liquid.

Next, another modified movable plug 4 of liquid holder 1 is described with reference to FIGS. 3A to 3C. FIGS. 3A to 3C illustrate Modification Example 1 of the movable plug. FIG. 3A is a sectional view of movable plug 10 in a normal state. FIG. 3B is a sectional view of movable plug 10 in a first deformation state. FIG. 3C is a sectional view of movable plug 10 in a second deformation state. In this Modification Example 1, the constitution other than movable plug 10 is the same as that of the liquid holder 1 illustrated in FIGS. 1A to 1C. Therefore, the illustration of the entire constitution is omitted in FIGS. 3A to 3C.

In this Modification Example, reservoir 3 is made of glass and has an inside diameter of 6 mm, and a length of 45 mm. Movable plug 10 is made of butyl rubber having rubber hardness of 40 degrees, an outside diameter of 6.1 mm, and a length of 5 mm (e.g., the lateral width in FIG. 3A). Membrane (second member) 10a of movable plug 10 has a thickness of 0.5 mm. Liquid 5 is purified water. The ejection is conducted at a driving voltage of 12 V, and a driving frequency of 25 kHz. As the result of ejection of liquid 5 from liquid holder 1 under the above-mentioned conditions, the pressure differential was reduced like that shown in FIG. 2A, characteristic of the present invention.

Before ejection of liquid 5 from liquid holder 1, or in an initial stage of the ejection, the pressure differential is not induced between the inside and outside of liquid holder 1 (at an approximately equal pressure), and movable plug 10 is in a state illustrated in the sectional view of FIG. 3A. In this state, the internal pressure in liquid holder 1 is balanced with the external pressure, and membrane 10 which will serve to reduce the pressure differential to be less than the pressure differential for initiating the movement of movable plug 10 is kept in a flat state, neither convexed nor concaved.

When the pressure in liquid holder 1 becomes negative relative to the outside by ejection of liquid 5 from liquid holder 1, membrane 10a is deformed toward the inside of reservoir 3 (leftward in FIG. 3B) when viewed from the front side of FIG. 3B. This deformation state corresponds to the time period (a-2) in FIG. 2A. With further progress of the ejection, membrane 10a is depressed to the deformation limit, and thereafter the pressure inside liquid holder 1 decreases again since the membrane cannot be deformed more.

On the other hand, when the pressure in liquid holder 1 becomes positive, for example, during storage, membrane 10a bulges out of liquid holder 1 (rightward in FIG. 3C when viewed from the front side of FIG. 3C). This deformation state can arise in an atmospheric pressure in an international passenger plane during takeoff. For example, in the case where the pressure of 1000 HP before the takeoff is decreased in about 20 minutes after the takeoff to 790 HPa, the pressure change of 210 HPa (=21 kPa) makes the atmospheric pressure outside liquid holder 1 negative relative to the pressure in liquid holder 1. Thereby, bubbles can often be formed in liquid 5 by liberation of the dissolved gas. The volume change caused by the gas liberation allows the movable plug 10 to slide to reduce the pressure differential remarkably.

The main parameter affecting the shape change of membrane 10a as illustrated in FIGS. 3B and 3C is the thickness of membrane 10a itself. That is, the thinner the membrane 10a, the larger can be the extent of the deformation of membrane 10a. However, the decrease of the membrane thickness will increase the gas permeability and water vapor permeability correspondingly. Therefore the thickness of membrane 10a should be adjusted to meet the use of liquid holder 1.

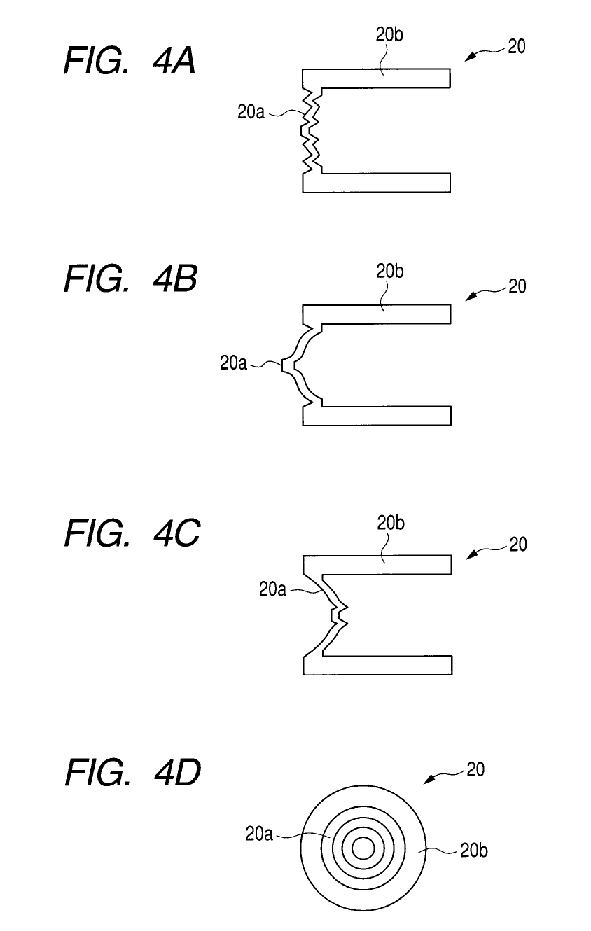

Next, another Modification Example 2 of the above-mentioned modified movable plug 4 is described with reference to FIGS. 4A to 4D. FIGS. 4A to 4D illustrate Modification Example 2 of movable plug 4. FIG. 4A is a sectional view of movable plug 20 in a normal state. FIG. 4B is a sectional view of movable plug 20 in a first deformation state. FIG. 4C is a sectional view of movable plug 20 in a second deformation state. FIG. 4D illustrates a view of movable plug 20 taken from the right side of FIG. 4A. In this Modification Example 2, the constitution other than movable plug 20 is the same as that of the liquid holder 1 illustrated in FIG. 1A to 1C. Therefore the illustration of the entire constitution is omitted in FIGS. 4A to 4C.

The movable plug 20 illustrated in FIGS. 4A to 4D, as an example, is improved to increase the possible deformation of membrane (a second member) 20a as the pressure differential-reducing member for increase of the extent of reduction of the pressure differential (e.g., the time for amount per second, or the repeating cycle time for every ejection). Movable plug 20 illustrated in FIGS. 4A to 4D is different from that of the above-mentioned movable plug 4 in the shape of membrane 4a and membrane 20a. Membrane 20a is regularly corrugated concentrically as illustrated in FIGS. 4A and 4D. FIG. 4B illustrates deformation of the membrane by a negative pressure in the liquid holder 1 (on the left side in FIG. 4B) relative to the outside thereof (on the right side in FIG. 4B). FIG. 4C illustrates deformation of the membrane at a positive pressure in the liquid holder 1 (on the left side in FIG. 4C) relative to the outside thereof.

As described above, in deformation of membrane 20a, the corrugated portion is expanded or contracted. Thereby, the deformation range can be made larger than that of membrane 4a having no corrugation to broaden the range of the allowable pressure differential. In other words, at a normal state, membrane 20a is in a folded state, and when a pressure differential is caused between the inside and outside of liquid holder 1, membrane 20a expands or constricts larger in comparison with membrane 4a to enlarge the range of pressure differential reduction.

In Modification Example 1 illustrated in FIG. 3A and Modification Example 2 illustrated in FIG. 4A, main sliding portion (first member) 10b, 20b is in a shape of a hollow cylinder. The hollow of main sliding portion 10b or 20b improves the responsiveness of movable plug 10, 20 to the pressure change inside liquid holder.

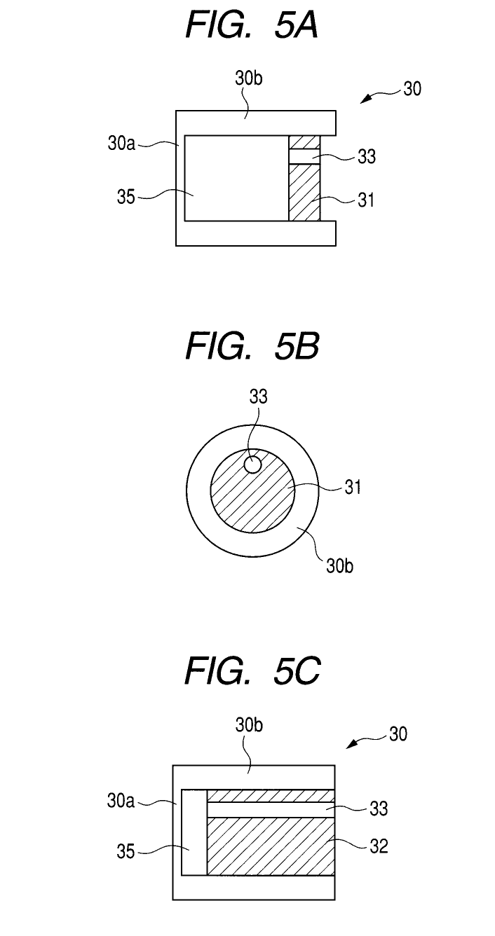

Modification Example 3 of movable plug 4 is described with reference to FIGS. 5A to 5C. FIGS. 5A to 5C illustrate Modification Example 3 of movable plug 4. FIG. 5A is a sectional view of movable plug 30 having spacer 31 placed in the hollow of main sliding portion 30b. FIG. 5B is a side view taken from the right side of FIG. 5A. FIG. 5C is a sectional view of another modification example of the movable plug. In this Modification Example 3, the constitution except movable plug 30 is the same as liquid holder 1 illustrated in FIGS. 1A to 1C. Therefore, the redundant description of the same constitution is omitted in FIGS. 5A to 5C.

Movable plug 30 has spacer 31 in the hollow of main sliding portion (first member) 30b. This spacer 31 is in a circular shape viewed from the right side in FIG. 5A as illustrated in FIG. 5B, and is in a disk shape having a thickness in the depth direction in the cylinder. This spacer 31 is preferably in a circular shape to come into contact with the inside wall face of main sliding portion 30b with a uniform contact force. Spacer 31 is placed in contact with the inside peripheral face of main sliding portion 30b at a suitable contact pressure to inside peripheral face of main sliding portion 30b. Thereby main sliding portion 30b is supported from the inside. The hollow in main sliding portion 30b and spacer 31 are circular in shape viewed from the right side in FIG. 5A. Therefore, the pressing force is applied by spacer 31 nearly uniformly with balance to main sliding portion 30b.

If spacer 31 is made of an air-tight material, the volume of the air in room 35 surrounded by movable plug 30 and spacer 31 changes in correspondence with the temperature, which affects the movability of movable plug 30. To prevent the influence of the air state in room 35 on movable plug 30, air hole 33 is preferably formed through spacer 31 as illustrated in FIGS. 5A to 5C. For example, without providing air hole 33, expansion of the air in room 35 increases the pressing force of movable plug 30 against main sliding portion 30b to retard the movement of movable plug 30. However, air hole 33 formed as illustrated in FIGS. 5A to 5C allows release of the increased portion of the air caused by expansion of the air in room 35 not to retard the movement of movable plug 30.

When spacer 31 is made from an air-permeable material, the above-mentioned air hole 33 need not be provided. An example is a sponge filter of a three-dimensional structure.

In the above description, spacer 31 is placed in the hollow of main sliding portion 30b. The thickness of the spacer (the lateral width in the front view of FIG. 5A) is not limited to that of the above-mentioned spacer 31. For example, the thickness may be like that of spacer 32 illustrated in FIG. 5C in the range not to interfere the swelling of membrane (second member) 30a (swelling rightward in FIG. 5C).

Spacer 31 as illustrated in FIG. 5A may be provided in plurality in the hollow of main sliding portion 30b for securing the rigidity of main sliding portion 30b (not shown in the drawings).

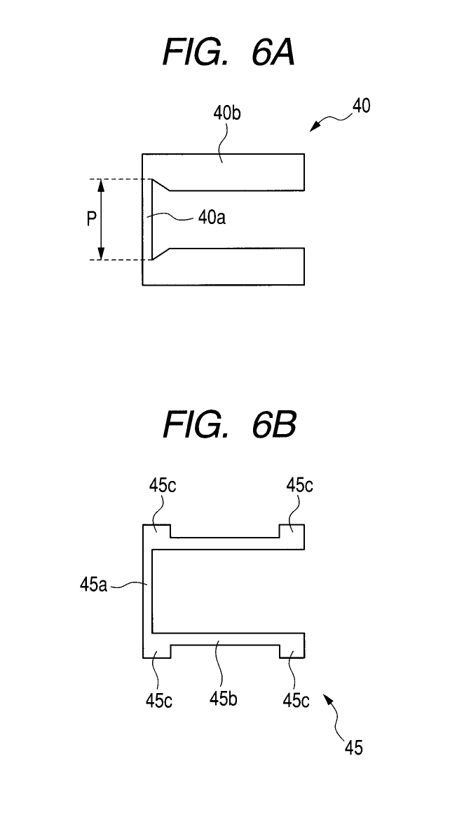

The movable plug having a hollow in main sliding portion 10b-30b like the ones in the above Modification Examples 1-3 may have main sliding portion 40b of a thick-wall structure to ensure the rigidity of main sliding portion (first member) 40b like that illustrated in FIG. 6A. Such a movable plug has preferably membrane 40a made thinner suitably to achieve the high performance of the pressure differential-reduction. With the thicker main sliding portion, a groove may be formed along the joint portion between expandable face P of membrane 40a and main sliding portion 40b to secure a room for expansion and contraction of the membrane.

The end 45c of main sliding portion 45b may have a thick-wall structure having an annular projection as illustrated in FIG. 6B. The sectional shape of end portion 45c (the shape in the front view in FIG. 6B) may be rectangular or trapezoidal. Further, the edge thereof may be rounded to adjust the pressure for initiating the movement of movable plug 45.

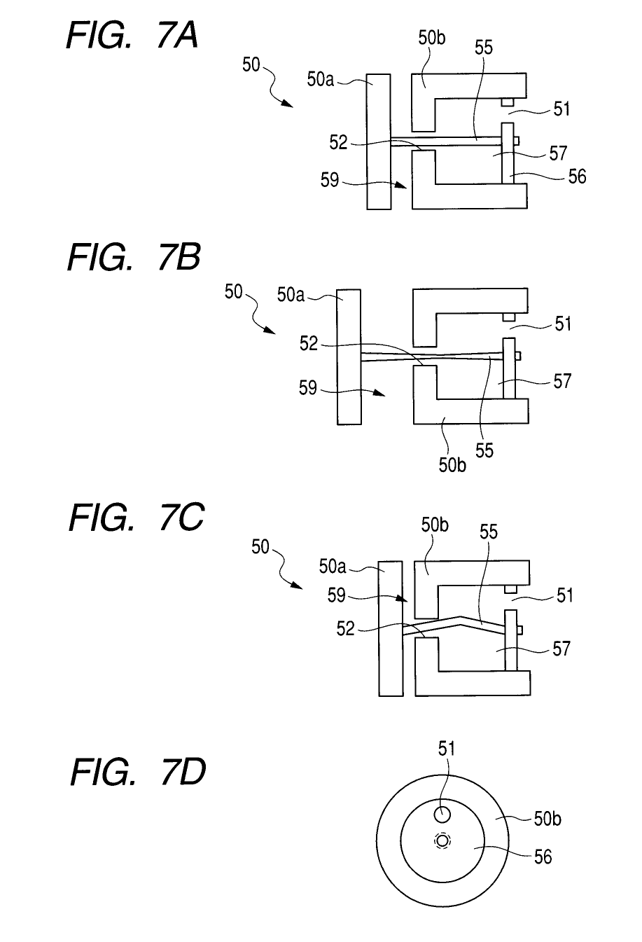

Modification Example 4 of the above-mentioned modified movable plug 4 is described with reference to FIGS. 7A to 7D. FIGS. 7A to 7D illustrate Modification Example 4 of movable plug 4. FIG. 7A is a sectional view of movable plug 50 in a normal state. FIG. 7B is a sectional view of movable plug 50 in a first deformation state. FIG. 7C is a sectional view of movable plug 50 in a second deformation state. FIG. 7D is a side view of movable plug 50 taken from the right side of FIG. 7A. In this Modification Example 4, the constitution other than movable plug 50 is the same as that of the liquid holder 1 illustrated in FIGS. 1A to 1C. Therefore, the illustration of the entire constitution is omitted in FIGS. 7A to 7D.

In movable plug 50 in FIGS. 7A to 7D, membrane (second member) 50a for reducing the pressure differential and main sliding portion (first member) 50b of movable plug 50 are connected into one body by connector 55 and connector support 56. Membrane 50a is circular when viewed from the left side or the right side in FIG. 7A in a disk shape. Main sliding portion 50b is circular when viewed from the left side or the right side in FIG. 7A, being nearly cylindrical, and has empty room 57 therein.

Main sliding portion 50b has through-hole 52 at the center of the wall at the front end (at the left end in FIG. 7A) thereof, and has through-hole 51 at connector support 56 on the wall of the rear side. Connector 55 connects membrane 50a with connector support 56 formed in main sliding portion 50b through the hole 52. Through-hole 51 serves as an air hole for communicating the room 57 of main sliding portion 50b with the outside of main sliding portion 50b.

FIG. 7A illustrates a normal state of movable plug 50 placed in reservoir 3, in which state no atmospheric pressure differential is caused between the inside and outside of reservoir 3. FIG. 7B illustrates the state in which membrane is displaced maximally into reservoir 3 with progress of liquid ejection through ejection outlet 7 (on the left side in the drawings) to cause a negative pressure in the reservoir 3 in comparison with the external pressure outside reservoir 3. The extent of reduction of the pressure by membrane 50a (including the distance of the displacement, the time for the displacement, and repetition number of the displacement) is controlled by adjusting the boldness and hardness of connector 55. The thinner and softer the connector, the larger is the elongation, whereas the thicker and harder the connector, the smaller is the elongation of the connector. The pressure for initiation of the movement of membrane 50a can be controlled by the contact area to reservoir 3, the compression degree in setting to the reservoir 3, the hardness of the material (elasticity) of membrane 50a, and so forth. At the maximum displacement of membrane 50a, further increase of the negative pressure in reservoir 3 initiates movement of the entire of movable plug 50 including main sliding portion 50b as if it is pulled by membrane 50a.

FIG. 7C illustrates membrane 50a pushed by liquid 5 in reservoir 3 by a positive internal pressure relative to the external pressure (maximally swollen state when viewed from the outside of reservoir 3). Since connector 55 is allowed to shrink or is bent in this state, connector 55 may be formed initially in a curved shape for ease of the bending. If space (gap) 59 between membrane 50a and main sliding portion 50b is tightly closed, the enclosed air can expand or contract to affect the movement of movable plug 50. Therefore, the aforementioned through-hole 51 on connector support 56 is necessary.

In the aforementioned Modification Examples 1-3, the member for reducing the pressure differential (membrane 10a, 20a, or 30a) constitutes a part of the movable plug (movable plug 10, 20, or 30), which may limit the freedom in production or design. However, in this Modification Example 4, membrane 50a and main sliding portion 50b can be designed independently in the material, shape, and hardness thereof. Membrane 50a and main sliding portion 50b can be produced in integration at a low production cost, but may be produced separately and combined later. Connector-support 56 is preferably formed in a simple structure in integration with connector 55. For example, one end of connector 55 is formed in a hook shape or in a J-shape, and a hook-receiving structure is provided on connector-support 56. Otherwise, main sliding portion 50b and connector-support 56 are connected, for example, by providing an annular groove along the inside periphery of main sliding portion 50b and fitting thereto connector support 56 having a diameter larger than the inside diameter of main sliding portion 50b by the depth of the groove.

The aforementioned membrane 50a, connector 55, and connector support 56 can be combined in two ways. In one way, membrane 50a and connector 55 are integrated into one body, and hooked to connector support 56. In another way, connector 55 and connector support 56 are integrated into one body, and hooked to membrane 50a.

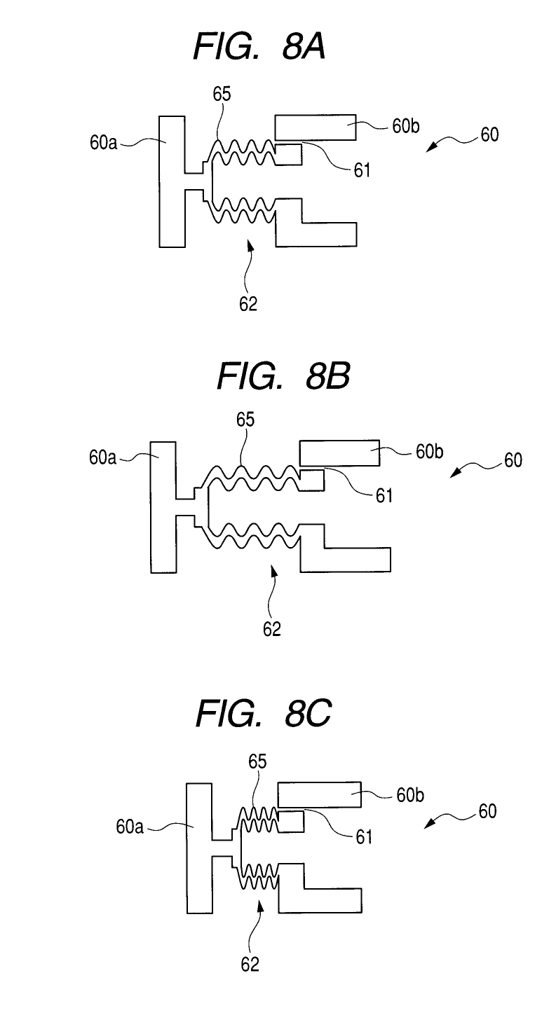

Modification Example 5 of the above-mentioned modified movable plug 4 is described with reference to FIGS. 8A to 8C. FIGS. 8A to 8C illustrate Modification Example 5 of movable plug 4. FIG. 8A is a sectional view of movable plug 60 in a normal state. FIG. 8B is a sectional view of movable plug 60 in a first deformation state. FIG. 8C is a sectional view of movable plug 60 in a second deformation state. In this Modification Example 5, the constitution other than movable plug 60 is the same as that of the liquid holder 1 illustrated in FIGS. 1A to 1C. Therefore, the illustration of the entire constitution is omitted in FIGS. 8A to 8C.

Movable plug 60, illustrated in FIGS. 8A to 8C, is constituted of membrane (second member) 60a for reducing the pressure differential, and main sliding portion (first member) 60b of movable plug 60 which are connected by connector 65 in integration. Connector 65 in this Example is in a shape of bellows. Membrane 60a and main sliding portion 60b are circular when viewed from the left side (or from the right side) in FIGS. 8A to 8C, and is inserted into reservoir 3 to fit uniformly to the inside wall of cylindrical reservoir 3. In the upper portion in the front view of FIG. 8A, through-hole 61 is formed which serves as an air hole for communication of the room (gap) 62 between membrane 60a and main sliding portion 60b with the outside air.

FIG. 8A illustrates a normal state of movable plug 60 placed in reservoir 3, in which state no atmospheric pressure differential is caused between the inside and outside of reservoir 3. FIG. 8B illustrates the state in which membrane 60a is displaced maximally into reservoir 3 with progress of liquid ejection through ejection outlet 7 (placed on the left side in the drawings) to cause a negative pressure in the reservoir 3 in comparison with the external pressure. The extent of reduction of the pressure by membrane 60a (including the distance of the displacement, the time for the displacement, and repetition number of the displacement) is controlled by adjusting the boldness and hardness of connector 65 similarly as in connector 55 illustrated in FIG. 7A.

FIG. 8C illustrates membrane 60a pushed by liquid 5 in reservoir 3 by a positive pressure relative to the external pressure (maximally swollen state when viewed from the outside of reservoir 3). If space 62 between membrane 60a and main sliding portion 60b is tightly closed, the enclosed air can expand or contract to affect the movement of movable plug 60. Therefore, the aforementioned through-hole 61 on connector support 56 is necessary for air communication. Since movable plug 60 in this Example is constituted of the same material in its entirety, the pressure for causing the movement of membrane 60a can be set by adjusting the sliding area in contact with reservoir 3.

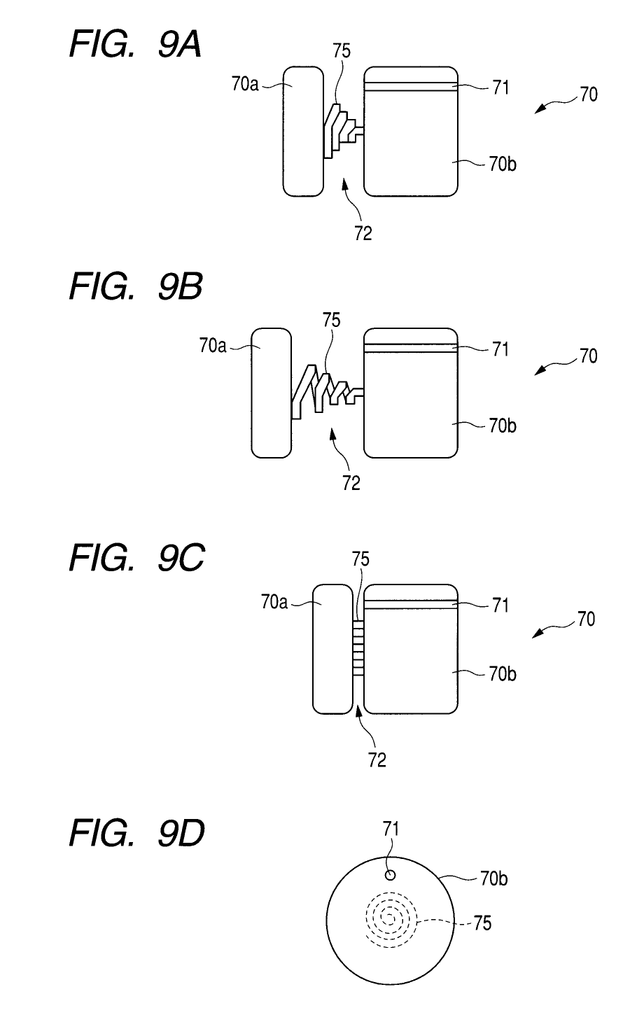

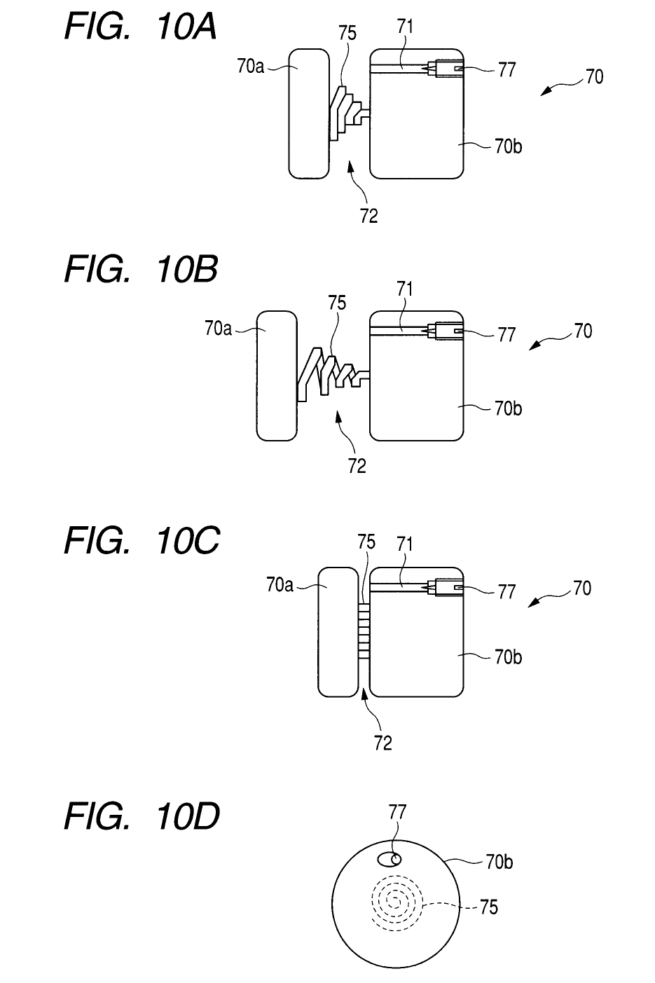

Modification Example 6 of the above-mentioned modified movable plug 4 is described with reference to FIGS. 9A to 9D. FIGS. 9A to 9D illustrate Modification Example 6 of movable plug 4. FIG. 9A is a sectional view of movable plug 70 in a normal state. FIG. 9B is a sectional view of movable plug 70 in a first deformation state. FIG. 9C is a sectional view of movable plug 70 in a second deformation state. FIG. 9D illustrates a view of movable plug 70 taken from the right side of FIG. 9A. In this Modification Example 6, the constitution other than movable plug 70 is the same as that of the liquid holder 1 illustrated in FIGS. 1A to 1C. Therefore the illustration of the entire constitution is omitted in FIGS. 9A to 9C.

Movable plug 70, illustrated in FIGS. 9A to 9D, is constituted of membrane (second member) 70a for reducing the pressure differential, and main sliding portion (first member) 70b of movable plug 70 which are connected by connector 75 in integration. Connector 75 in this Example is in a shape of a spiral. Membrane 70a and main-sliding portion (first member) 70b has the corners rounded (edges in the portions in contact with reservoir 3) as illustrated in the front view of FIG. 9A. Membrane 70a and main sliding portion 70b are circular when viewed from the left side (or from the right side) in FIGS. 9A to 9C, and is inserted into reservoir 3 to fit uniformly to the inside wall of cylindrical reservoir 3. In the upper portion in front view of FIG. 9A, through-hole 71 is formed between connector 75 and main sliding portion 70b. This through-hole 71 serves as an air hole for communication of the space (gap) 72 between membrane 70a and main sliding portion 70b with the outside air.

FIG. 9A illustrates a normal state of movable plug 70 placed in reservoir 3, in which state no atmospheric pressure differential is caused between the inside and outside of reservoir 3. FIG. 9B illustrates the state in which membrane 70a is displaced maximally into reservoir 3 with progress of liquid ejection through ejection outlet 7 (on the left side in the drawings) to cause a negative pressure in the reservoir 3 in comparison with the external pressure. Membrane 70a is moved leftward with elongation of connector 75 folded in a spiral state. The extent of reduction of the pressure by membrane 70a (including the distance of the displacement, the time for the displacement, and repetition number of the displacement) is controlled by adjusting the boldness and hardness of connector 75, and the winding strength of the spiral.

FIG. 9C illustrates membrane 70a pressed by liquid 5 in reservoir 3 by a pressure positive in comparison with the external pressure (maximally bulging state viewed from the outside of reservoir 3). If space 72 between membrane 70a and main sliding portion 70b is tightly closed, the enclosed air can expand or contract to affect the movement of movable plug 70. Therefore, the aforementioned through-hole 71 is necessary for air communication as shown in the drawings. Movable plug 70 in this Embodiment is constituted of the same material in its entirety. Therefore the pressure for initiating the movement of membrane 70a can be set by adjusting the sliding contact area with reservoir 3.

Modification Example 7 of the above-mentioned movable plug 4 is described with reference to FIGS. 10A to 10D. FIGS. 10A to 10D illustrate a movable plug of Modification Example 7 based on Modification Example 6. FIG. 10A is a sectional view of movable plug 70 in a normal state. FIG. 10B is a sectional view of movable plug 70 in a first deformation state. FIG. 10C is a sectional view of movable plug 70 in a second deformation state. FIG. 10D illustrates a view of movable plug 70 taken from the right side in FIG. 10A. In this Modification Example 7, the constitution other than air flow controller 77 is the same as movable plug 70 illustrated in FIG. 9A to 9D. Therefore the redundant description on movable plug 70 is omitted here. Further, the constitution other than movable plug 70 is the same as that of the liquid holder 1 illustrated in FIGS. 1A to 1C. Therefore the illustration of the entire constitution is omitted in FIGS. 10A to 10C.

Movable plug 70 as illustrated in FIGS. 10A to 10D has air flow controller 77 at the opening of through-hole 71 at the end of main sliding portion 70b (e.g., at the right end in the front views of FIGS. 10A to 10D). This air flow controller 77 is generally called a speed controller, and lowers an operation speed of a part in pneumatic operation apparatus. In this Modification Example, this controller enables fine control of the operation pressure for initiating the movement of membrane 70a to raise the operation pressure. Therefore, with movable plug 70 having air flow controller 77 illustrated in FIGS. 10A to 10D, the operation pressures for membrane 70a and main sliding portion 70b are raised to enable increase of the operation speeds.

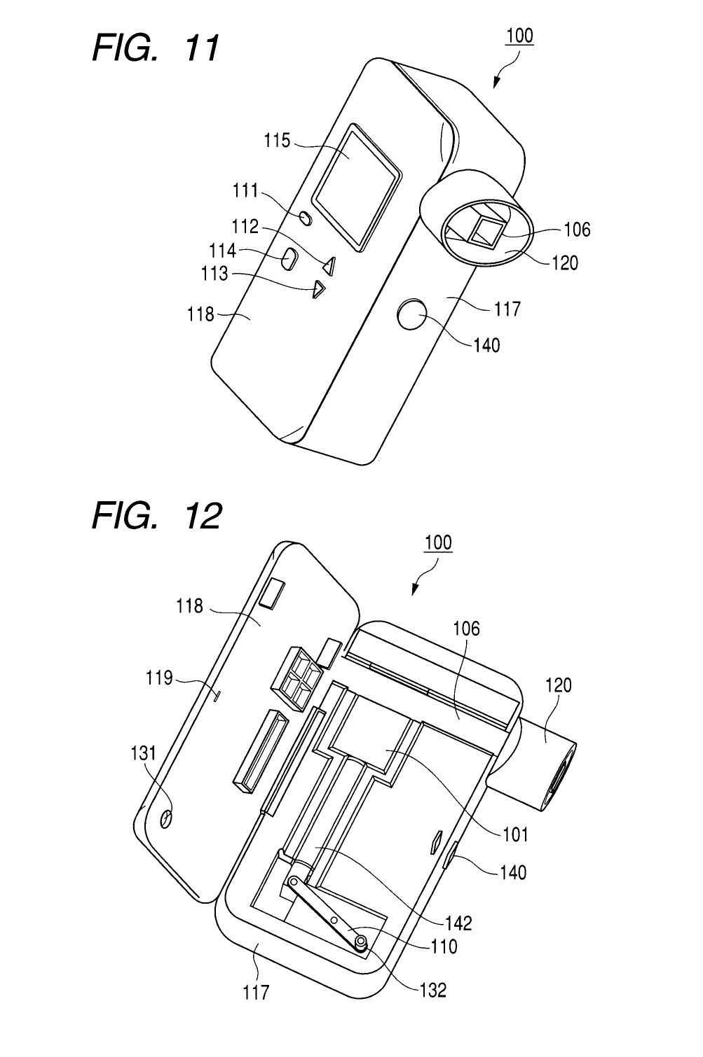

Next, a specific example of the use of liquid holder 1 of this Embodiment is described with reference to FIGS. 11 and 12. FIG. 11 is a schematic sectional view of an example of an apparatus 100 for medical solution ejection, employing liquid holder 1 of the present invention for inhalation of a medical solution by a user. FIG. 12 is a perspective view of inhalation apparatus 100 with access cover 118 opened

In FIGS. 11 and 12, inhalation apparatus 100 has a casing constituted of housing case 117 and access cover 118. The case and the cover are locked by engaging hook 119 with a hook receiver, and function together with spring-energized unlocking button 140. For opening access cover 118, unlocking button 140 is pressed to unlock the hooking. Thereby the access cover 118 is opened by the force of a spring (not shown in the drawing) energized for the opening.

Housing case 117 comprises inhalation port 120 having air flow path 106, unlocking button 140 for releasing the lock of access cover 118. Access cover 118 has display unit 115 for displaying an administration amount, an administration time, an error sign, and so forth; menu-changing button 111 for setting by a user: up-directing button 112, down-directing button 113; and setting button 114. Incidentally, the above-mentioned inhalation port 120 is called also a mouthpiece.

FIG. 12 illustrates inhalation apparatus 100 with access cover 118 opened. With access cover 118 opened, ejection head 101 as the liquid ejection assembly and liquid tank 142 as the medical solution container are visible. Both of ejection head 101 and medical solution tank 142 are demountable from the main body of the apparatus. Ejection head 101 ejects the medical solution into air flow path 106. The user can inhale the medical solution ejected into air flow path 106 by breathing in the air through inhalation port 120. In inhalation apparatus 100 of this embodiment, inhalation port 120 and air flow path 106 are combined into one body.

Inhalation port 120 may be discarded after one inhalation or the used port after the inhalation may be reused after cleaning. Ejection head 101 and liquid tank 142 are exchanged when the amount of the medical solution in liquid tank 142 becomes less than the one inhalation dose. For example, the apparatus has a counter for counting the amount of the ejected medical solution. This counter is capable of counting the remaining amount of the liquid. Thereby, the time of container exchange can be notified to the user, the user is urged to exchange the drug container, or the ejection can be interrupted until the completion of the exchange. Ejection head 101 and liquid tank 142, after mounting, is connected to ejection head 101 by pushing the liquid tank 142 by connection lever 110 toward ejection head 101 to form a liquid flow path for introducing the medical solution from liquid tank 142 into ejection head 101.

Access cover 118 has, on its reverse face, a connection lever-locking hole 131 (FIG. 12). With the access cover 118 closed, knob 132 of connection lever 110 fits into connection lever-locking hole 131, whereby ejection head 101 and liquid tank 142 are kept connected unless access cover 118 is opened. Thereby the disconnection of liquid tank 142 from ejection head 101 is prevented during carrying in a bag or the like.

As described above, liquid holder 1 of First Embodiment of the present invention, has stopper 2 through which outlet 7 is formed for discharging the liquid 5 held therein, and movable plug 4 for reducing the pressure differential between the inside and outside of liquid holder 1. Movable plug 4 has main sliding portion 4b (or main sliding portion 10b-70b) which moves to reduce a prescribed first pressure differential or higher; and membrane 4a (or membrane 10a-70a) for reducing the second pressure differential within a prescribed level. Thereby the pressure differential between the inside and outside of liquid holder 1 can be kept to be relatively smaller, and the decrease of the ejection performance of liquid holder 1 can be made smaller than that of conventional ones.

Membrane 4a (or membrane 10a-40a) and main sliding portion 4b (or main sliding portion 10b-40b) are formed in one body, and move together to reduce a prescribed first pressure differential (−10 kPa). Membrane 4a (or membrane 10a-40a) itself deforms to reduce the second pressure differential less than the prescribed first pressure differential. Thereby movable plug 4 can be produced in a simple structure at a relatively low cost, and the parts can be controlled readily owing to one-body structure of movable plug 4.

Membrane 50a (or membrane 60a, 70a) and main sliding portion 50b (or main sliding portion 60b, 70b) are connected by strechable connector 55 (or connector 65, 75) to move together to reduce the prescribed pressure differential (−10 kPa). Membrane 50a (or membrane 60a, 70a) reduces the pressure differential in the range smaller than the prescribed level by changing the distance from main sliding portion 50b (or main sliding portion 60b, 70b). Since membrane 50a, for example, is movable within liquid holder 1, the time and amount of the prescribed pressure differential can be designed for reduction of time and amount in a relatively wide range.

Main sliding portion 50b, for example, has air hole 51 for air communication of the gap between membrane 50a and main sliding portion 50b to the outside of liquid holder 1. This air hole serves to make the atmospheric pressure in room 59 between membrane 50a and main sliding portion 50b equal to the atmospheric pressure outside liquid holder 1 to make smooth the displacement of membrane 50a and main sliding portion 50b.

### Second Embodiment

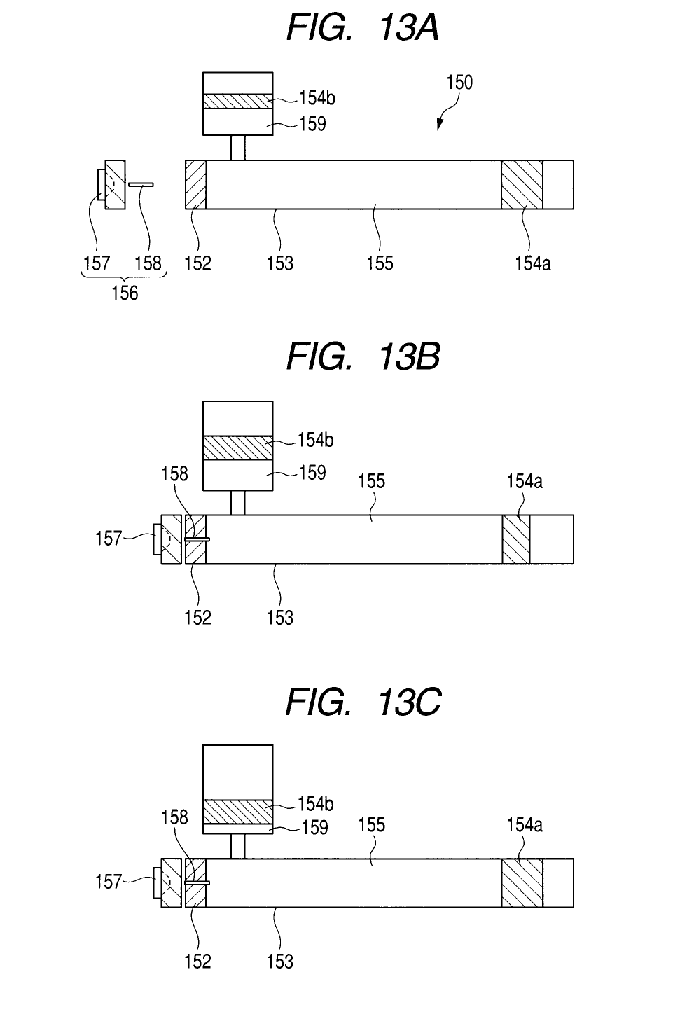

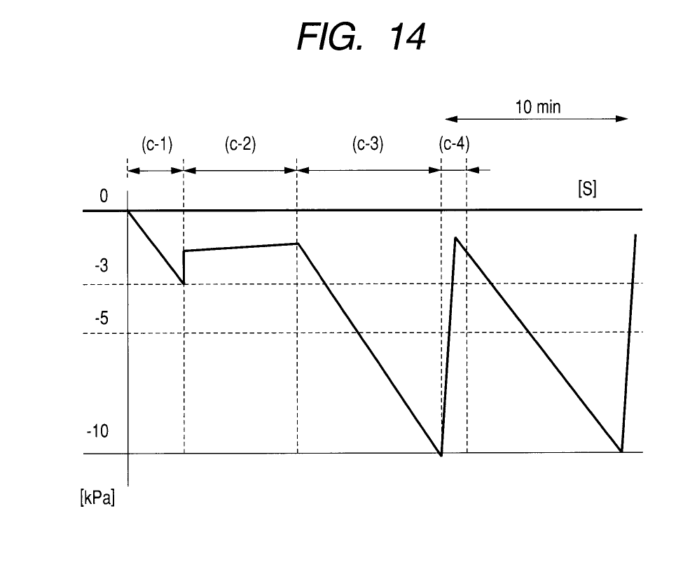

Liquid holder 150 of Second Embodiment of the present invention is described with reference to FIGS. 13A to 13C and FIG. 14. FIGS. 13A to 13C illustrates schematically the constitution of the liquid holder in Second Embodiment. FIG. 13A is a schematic drawing before connection of ejection head 156. FIG. 13B illustrates schematically a first state after connection of ejection head 156. FIG. 1C illustrates schematically a second state after connection of ejection head 156. FIG. 14 is a graph showing change with time of the pressure in liquid holder 150 during ejection of the liquid.

Liquid holder 150 comprises first reservoir 153 and second reservoir 159 for holding liquid 155, and stopper (outlet formation part) 152, first movable plug (first member) 154a, and second movable plug (second member) 154b as illustrated in FIG. 13A. First reservoir 153 and second reservoir 159 are respectively made from a rigid material (e.g., glass) in a cylindrical shape. Stopper 152 closes the lengthwise end of cylindrical first reservoir 153, and first movable plug 154a closes the other lengthwise end. Second reservoir 159 is connected to the side of first reservoir 153. Liquid 155 is enclosed therein by second movable plug 154b.

Ejection head 156 having communication needle 158 is connectible to stopper 152 by inserting communication needle 158 from the position confronting stopper 152 outside liquid holder 150. Ejection head 156 connected to liquid holder 150 can eject liquid 155 contained in liquid holder 150 through ejection outlet 157. Thus, ejection outlet 157 for ejecting liquid 155 can be formed through stopper 152. Ejection head 156 has the same constitution as ejection head 6 in First Embodiment, and ejection head 156, ejection outlet 157, and communication needle 158 in this Embodiment correspond respectively to ejection head 6, ejection outlet 7, and communication needle 8 in Embodiment 1. Therefore, description thereof is omitted.

Liquid holder 150 of this Embodiment is different characteristically from the one of Embodiment 1 in that a second movable plug 154b is provided, in addition to first movable plug 154a, for reducing the pressure differential below the level for initiating the displacement of first movable plug 154a. The inside diameter of second movable plug 154b and the inside diameter of second reservoir 159 are respectively larger than the inside diameter of first movable plug 154a and the inside diameter of first reservoir 153. Therefore, the sectional area in the diameter direction of second movable plug 154b is larger than that of first movable plug 154a. Therefore, the negative pressure in first reservoir 153 and second reservoir 159 applies a stronger force to second movable plug 154b than to first movable plug 154a to cause displacement of second movable plug 154b by a less pressure differential.

In the constitution of liquid holder 150, first reservoir 153 is made of glass, and has an inside diameter of 6 mm, and a length of 45 mm. The first movable plug 154a is made of a butyl rubber having a rubber hardness of 40 degrees, and has an outside diameter of 6.1 mm and a length of 5 mm. Second reservoir 159 is made of glass, and has an inside diameter of 12 mm, and a length of 10 mm. Second movable plug 154b is made of a butyl rubber having a rubber hardness of 40 degrees, and has an outside diameter of 12.1 mm and a length of 5 mm. Purified water is used as liquid 155.

As an example, the behavior of the above-mentioned first movable plug 154a and second movable plug 154b was investigated under the pressure change at landing of an international passenger plane. In landing of the international passenger plane, usually the atmospheric pressure changes from 770 HPa to 1020 HPa in about 26 minutes. The difference in the atmospheric pressure is 250 HPa (=25 kPa). The investigation shows reduction of the pressure differential like that indicated in the graph in FIG. 14.

The operation of liquid holder 150 is considered for time periods (c-1), (c-2), (c-3), and (c-4) shown in FIG. 14. The description below is based on this division of the time periods from (c-1) to (c-4).

FIG. 14 shows that, in the above-mentioned conditions, the atmospheric pressure outside liquid holder 150 increases at a rate of about 1 kPa/min, and three minute later, the pressure differential between the inside and outside of liquid holder 150 becomes −3 kPa (time period (c-1)).

At the internal pressure of −3 kPa, second movable plug 154b begins to move to reduce the pressure differential, which is smaller than the pressure differential for initiating the movement of first movable plug 154a to keep the pressure differential (time period (c-2)). With further decrease of the internal pressure in liquid holder 150, second movable plug 154b reaches the displacement limit. After the reach of the second movable plug 154b to the displacement limit for reducing the pressure differential, the internal pressure comes to decrease again at the same rate as that in time period (c-1) continually (see time period (c-3)).

With further decrease of the pressure in liquid holder 150, first movable plug 154a start to move when the internal pressure comes to be lower than the prescribed level at which first movable plug 154a start to move. Thereby, the pressure differential between the inside and outside of the holder is reduced until first movable plug 154a comes to stop (time period (c-4)). First movable plug 154a stops when the dynamic frictional force of first movable plug 154a becomes stronger than the driving force produced by the pressure differential.

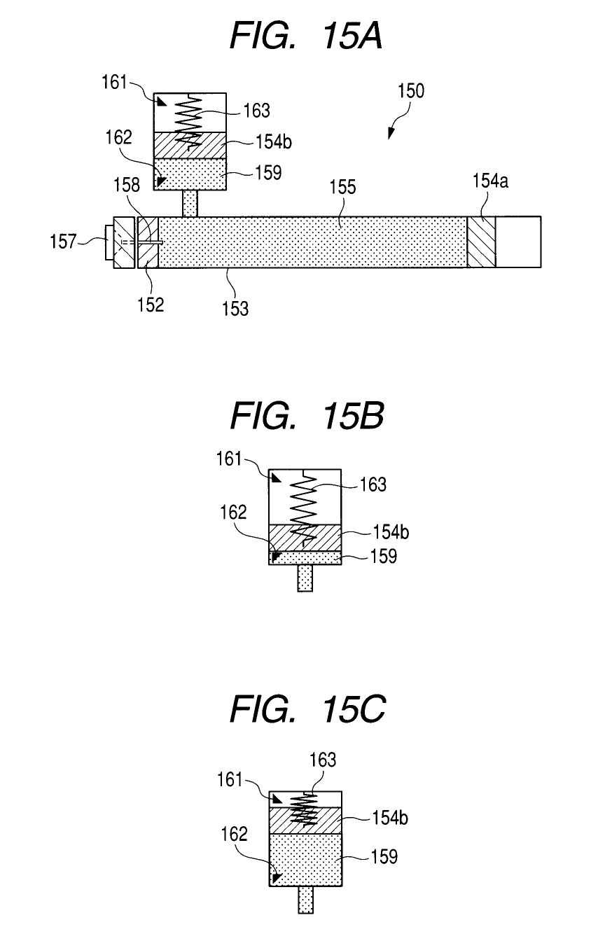

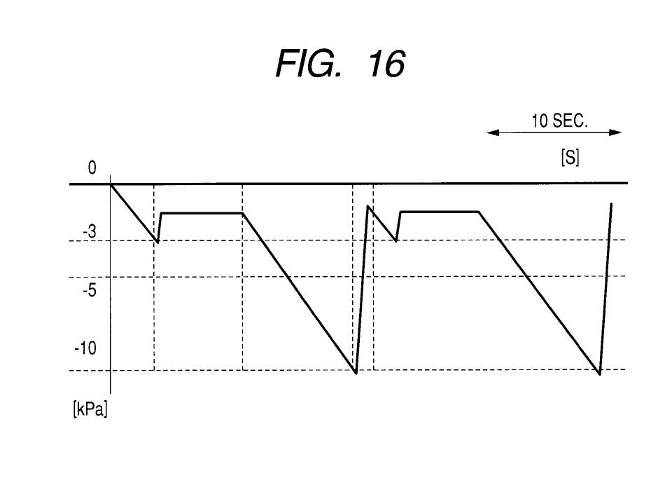

With liquid holder 150 illustrated in FIGS. 13A to 13C, the operation of reducing the pressure differential (i.e., operation for reducing the pressure differential at a level less than that for initiating the movement of first movable plug 154a) is conducted only once, and the above-mentioned operation of reducing the pressure differential can not be conducted further. The example illustrated in FIGS. 15A to 15C is improved to conduct repeatedly the reduction of the pressure differential at a less pressure differential.

Modification Example 1 of liquid holder 150 is described with reference to FIGS. 15A to 15C and FIG. 16. FIGS. 15A to 15C illustrate another liquid holder 150 of Modification Example 1. FIG. 15A is a sectional view of liquid holder 150 in a normal state. FIG. 15B is a sectional view thereof in a first state of second reservoir 159. FIG. 15C is a sectional view thereof in a second state of second reservoir 159. FIG. 16 is a graph showing a pressure change with time in liquid holder 150 with ejection of the liquid. In this Modification Example 1, the constitution is the same as the one of liquid holder 150 in FIGS. 13A to 13C except position-limiters 161, 162 and neutral-position recovery mechanism 163. Therefore, the redundant description thereof is omitted.

Liquid holder 150 in this Example has a rigid second reservoir 159 as illustrated in FIGS. 15A to 15C, in which are provided position-limiters 161, 162 for limiting the movable range of the second movable plug 154b, and neutral-position recovery mechanism 163 which connects second movable plug 154b to the top end of second reservoir 159 and brings second movable plug 154b to the neutral position. The term “neutral position” herein signifies the middle position between position-limiter 161 and position-limiter 162 in the vertical direction. An example of the neutral-position recovery mechanism is a spring. This modification example employs a spring as neutral-position recovery mechanism 163, and second movable plug 154b is placed, in the initial state, at the neutral position at which the neutral-position recovery mechanism 163 is in a natural state without elongation or compression.

Liquid ejection head 156 was connected to liquid holder 150, and liquid 155 was ejected through communication needle 158 and ejection outlets 157. Specifically, liquid ejection head 156 has 20000 fine ejection holes, and liquid 155 was ejected as liquid droplets for one second in an ejection amount of 30 μm/sec at a frequency of 30 kHz. With ejection of liquid 155, the amount of liquid 155 in liquid holder 150 decreased to cause a negative pressure in liquid holder 150 and a pressure differential between the inside and outside of the liquid holder. The above-mentioned one ejection cycle caused decrease of the internal pressure in liquid holder 150 by 1 kPa according to measurement with a manometer (not shown in the drawings).

With liquid holder 150 of this modification example, the ejection was conducted for 30 seconds under the above conditions. FIG. 16 shows the change of the internal pressure in the holder.

After the start of the ejection, the amount of the liquid in liquid holder 150 decreases to lower the internal pressure in liquid holder 150 to −3 kPa. When the internal pressure becomes lower than −3 kPa, second movable plug 154b starts to move (downward in front view in FIG. 15A) to keep the pressure inside liquid holder 150 at about −3 kPa. With continuation of the ejection, second movable plug 154b reaches the lower limit of the displacement to come to contact with position-limiter 162 with neutral-position recovery mechanism 163 lengthened maximally as shown in FIG. 15B.

As shown in FIG. 15B, after second movable plug 154b comes into contact with position-limiter 162, the internal pressure in liquid holder 150 begins to decrease again. When the internal pressure has come to be lower than −10 kPa, first movable plug 154a start to move. After the start of movement of first movable plug 154a until it is stopped next, the pressure differential between the inside and outside of liquid holder 150 is reduced. With the reduction of the pressure differential, the force of liquid 155 to flow from first reservoir 153 into second reservoir 159 and the energizing force of neutral-position recovery mechanism 163 allows second movable plug 154b to return to the neutral position in second reservoir 159. Incidentally, in FIG. 15C, second movable plug 154b is in contact with position-limiter 161 at the uppermost position of the displacement limit, and neutral-position recovery mechanism 163 is compressed maximally.

As described above, when first movable plug 154a is moved to reduce the pressure differential by a negative pressure in liquid holder 150, neutral-position recovery mechanism 163 allows second movable plug 154b to return from the lower limit of the displacement range as illustrated in FIG. 15B to the neutral position as illustrated in FIG. 15A. After this, the process of the reduction of a smaller pressure differential is started again by second movable plug 154b (the operation of reducing the pressure differential smaller than the pressure differential for initiating the movement of first movable plug 154a).

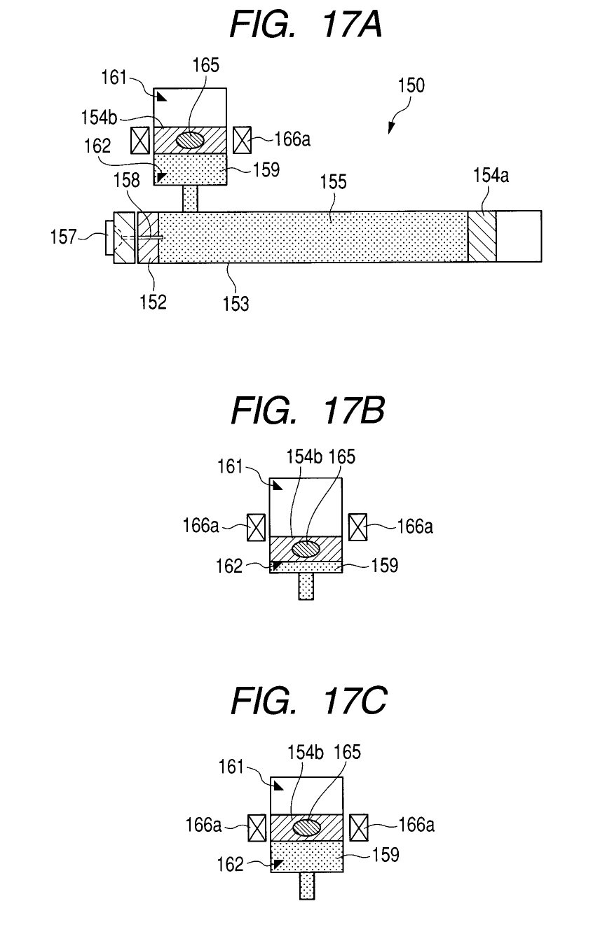

Another Modification Example 2 of liquid holder 150 is described with reference to FIGS. 17A to 17C. FIGS. 17A to 17C illustrate another liquid holder 150 of Modification Example 2. FIG. 17A is a sectional view of liquid holder 150 in a normal state. FIG. 17B is a sectional view thereof at a first state of second reservoir 159. FIG. 17C is a sectional view thereof at a second state of second reservoir 159. In this Modification Example 2, the constitution is the same as the one of liquid holder 150 in FIGS. 15A to 15C except attractable member 165 and electromagnet 166a. Therefore the redundant description thereof is omitted.

Liquid holder 150 of this modification example has attractable member (recovery means) 165 and electromagnet (recovery means) 166a in or near second reservoir 159. In the aforementioned Modification Example 1, a spring is employed as neutral-position recovery mechanism 163 for returning second movable plug 154b to the neutral position. The neutral-position recovery mechanism is not limited thereto, and may be a combination of an attractable member 165 and electromagnet 166a.

Attractable member 165 is a member which can be attracted by a magnetic force like that of a magnet, and is placed at the center in second movable plug 154b as illustrated in FIG. 17A. Electromagnet 166a is a coil which can be magnetized by electric current application, and is placed at the middle position in the height direction of second reservoir 159, namely at the neutral position in second movable plug 154b.

With liquid holder 150 of this modification example having liquid ejection head 156 connected thereto, ejection of liquid 155 causes a negative pressure in liquid holder 150, and correspondingly second movable plug 154b moves downward to reduce the pressure differential at the small pressure differential range, and reaches the lower limit position of second movable plug 154b to come to contact with position-limiter 162 as illustrated in FIG. 17B. In this state, second movable plug 154b can be returned to the neutral position by a magnetic force generated by application of electric current to electromagnet 166a as illustrated in FIG. 17C. In this Modification Example, liquid 155 is ejected for 30 second under the same conditions as in the above Modification Example 1. In the process of the ejection, the internal pressure in liquid holder 150 changes in the same manner as shown in FIG. 16.

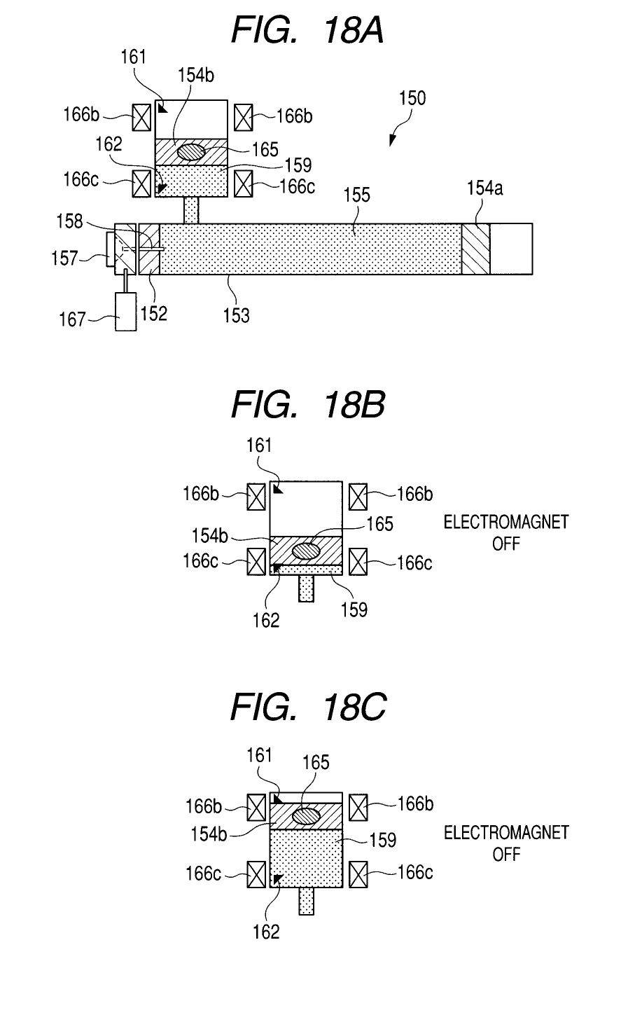

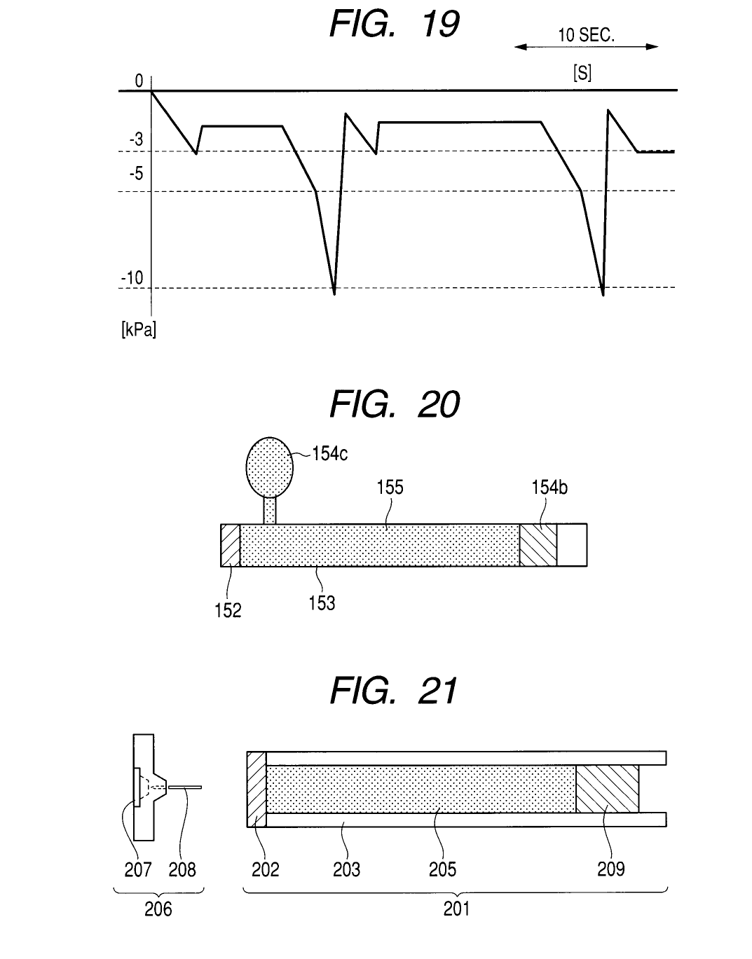

Modification Example 3 of liquid holder 150 is described with reference to FIGS. 18A to 18C and FIG. 19. FIGS. 18A to 18C illustrate another liquid holder 150 of Modification Example 3. FIG. 18A is a sectional view of liquid holder 150 in a normal state. FIG. 18B is a sectional view thereof at a first state of second reservoir 159. FIG. 18C is a sectional view thereof at a second state of second reservoir 159. FIG. 19 is a graph showing a change of the pressure with time in liquid holder 150 in the course of ejection of the liquid. In this Modification Example 3, the constitution is the same as the one of liquid holder 150 in FIGS. 17A to 17C except electromagnets 166b, 166c and pressure sensor 167. Therefore, the redundant description thereof is omitted.

In this Modification Example, the movement of second movable plug 154b is controlled to improve the reduction of the pressure differential by first movable plug 154a. Liquid holder 150 of this Example has electromagnets (recovery means) 166b, 166c to surround the outside periphery of second reservoir 159. Electromagnets 166b, 166c are constituted of coils which are magnetizable by electric current application, and are placed respectively around a top portion and around a bottom portion of second reservoir 159, or at the same heights as position-limiters 161, 162. Ejection head 156 of this Example has pressure sensor 167 for sensing the pressure in liquid holder 150. A control circuit (not shown in the drawing) turns on and off electromagnets 166b, 166c in accordance with the output signals emitted from this pressure sensor.

In ejection of liquid 155 from liquid holder 150 having liquid head 156 attached thereto, first movable plug 154a and second movable plug 154b are moved in accordance with the negative pressure caused in liquid holder 150. Before the ejection of liquid 155 from liquid ejection head 156, second movable plug 154b is placed at the neutral position as illustrated in FIG. 18A. In the course of ejection of liquid 155, second movable plug 154b is moved downward. When the second movable plug reaches the lower limit of the displacement range as illustrated in FIG. 18B, electromagnet 166b is turned on to bring second movable plug 154b upward to the upper limit of the displacement. In this Embodiment, liquid 155 is ejected for 30 second under the same conditions as in the aforementioned Modification Example 2. FIG. 19 shows the variation with time of the pressure in liquid holder 150 during the liquid ejection.

The above-mentioned timing of the turn-on of electromagnet 166b can be decided, for example, as follows. The internal pressure difference for initiating the movement of second movable plug 154b from the lower limit to the upper limit of the displacement range is measured by pressure sensor 167. This measured pressure change is represented by P1. Then from the pressure difference for initiating movement of first movable plug 154a, 10 kPa in this Example, the above calculated pressure differential P1 is subtracted. At the time when the pressure difference has come to the above calculated level (e.g., 10-P1), electromagnet 166b is turned on. Thereby the duration of instable ejection through liquid ejection head 156 can be shortened. Further, during the time of forcible movement of second movable plug 154b by electromagnet 166b, the ejection of liquid 155 through liquid ejection head 156 may be stopped. In this example, pressure sensor 167 is employed, but a pressure switch or the like may be employed instead.

Modification Example 4 of liquid holder 150 is described with reference to FIG. 20. FIG. 20 is a sectional view of another liquid holder 150 of Modification Example 4. In this Modification Example 4, the constitution is the same as the one of liquid holder 150 in FIGS. 13A to 13C except that flexible reservoir 154c is used in place of second movable plug 154b and second reservoir 159. Therefore, the redundant description thereof is omitted.

As illustrated in FIG. 20, liquid holder 150 of this Modification Example has flexible reservoir 154c connected to first reservoir 153 in place of second reservoir 159 shown in FIGS. 13A to 13C. Flexible reservoir 154c is made of a flexible material of the same quality as membrane 4a shown in FIG. 1. Flexible reservoir 154c encloses liquid 155 therein. With this constitution, the flexible reservoir 154c serves to reduce the pressure differential between the inside and outside of liquid holder 150 by contraction or recovery to the original state instead of using second movable plug 154b shown in FIGS. 13A to 13C.

In this example, the pressure for initiating the contraction of flexible holder 154c can be adjusted by the thickness, shape, or the like properties of flexible reservoir 154c. Thus, in this example, flexible reservoir 154c, which has a function of second reservoir 159 and second movable plug 154c in FIGS. 13A to 13C in one body, serves for reducing the pressure differential at a small pressure differential range. Thus the production cost can be lowered and the control of the parts can be made easier.

As described above, liquid holder 150 of Second Embodiment has stopper 152 for formation of outlet 157 discharging liquid 155 held therein. Liquid holder 150 has further first movable plug 154a for reducing a predetermined level of the pressure differential between the inside and outside of liquid holder 150, and second movable plug 154b for reducing the pressure differential below the predetermined level. Thereby the pressure differential between the inside and outside of liquid holder 150 can be maintained within a relatively narrow range, whereby the drop of ejection performance of liquid holder 150 can be decreased.

Second movable plug 154b has neutral-position recovery mechanism 163, which brings second movable plug 154b to the neutral state for reducing the second pressure differential less than a predetermined first pressure differential between the inside and outside of liquid holder 150 when the pressure differential is reduced to the predetermined level. Thereby, the process of reducing the second pressure differential less than the predetermined first level can be repeated with second movable plug 154b, even though first movable plug 154a and second movable plug 154b are not integrated into one body.

Liquid holder 150 has position-limiter 161, 162 for limiting the displacement range of second movable plug 154b. Thereby second movable plug 154b can be moved smoothly and repeatedly, and penetration of the outside air into the liquid holder 150 can be prevented.

As described above, according to First Embodiment and Second Embodiment, inhalation apparatus 100 has liquid holder 1 or 150, ejection head 6 or 156, and inhalation port 120 for inhalation of a liquid ejected from the above ejection head by a user. This inhalation apparatus 100 causes less deterioration in the ejection performance in comparison with conventional ones.

Inhalation apparatus 100 described with reference to FIGS. 11 and 12 for First Embodiment can employ suitably liquid holder 150 of Modification Examples 1-4 of Second Embodiment.

The liquid holder, and the inhalation apparatus employing the liquid holder are useful in the case where the pressure differential between inside and outside of the liquid holder should be kept smaller, and are useful for stable ejection of a medical solution.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2008-186810, filed Jul. 18, 2008, which is hereby incorporated by reference herein in its entirety.

1. A liquid holder for holding a liquid comprising:
an outlet formation part for formation of outlet port for discharging the liquid held in the liquid holder, and
a pressure differential-reducing member for reducing a first predetermined pressure differential between the inside and outside of the liquid holder;
the pressure differential-reducing member including a first member which moves for reducing the first predetermined pressure differential and a second member which reduces a second pressure differential less than the first predetermined pressure differential.

2. The liquid holder according to claim 1, wherein the first member and the second member are formed in one body, and move together when reducing the first predetermined pressure differential, the second member deforms to reduce the second pressure differential less than the first predetermined pressure differential.

3. The liquid holder according to claim 1, wherein the first member and the second member are connected by an expandable connector and move together when reducing the first predetermined pressure differential, and
the second member reduces the second pressure differential less than the first predetermined pressure differential by changing the distance from the first member.

4. The liquid holder according to claim 3, wherein the first member has an air hole for communicating a gap between the first member and the second member with the outside of the liquid holder.

5. The liquid holder according to claim 1, wherein the pressure differential-reducing member has a recovery means for bringing the second member to be ready for reducing the second pressure differential less than the first predetermined pressure differential at the time when the first predetermined pressure differential has been reduced by movement of the first member.

6. The liquid holder according to claim 1, wherein the pressure differential-reducing member has a position-limiter for limiting the range of displacement of the first member or the second member.

7. An inhalation apparatus, comprising
a liquid holder set forth in claim 1,
an ejection head for ejecting a liquid held in the liquid holder, and
a suction port for inhalation of the liquid ejected from the ejection head by a user.
